(19) United States
(12) Patent Application Publication
(10) Pub. No.: US 2002/0161338 A1
(43) Pub. Date: Oct. 31, 2002

(54) Catheters and catheter safety guards

(76) Inventor: Francis Peterson, Spooner, WI (US)

Correspondence Address: FOLEY AND LARDNER, SUITE 500, 3000 K STREET NW, WASHINGTON, DC, 20007, US

(21) Appl. No.: 10/097,117

(22) Filed: Mar. 14, 2002

Related U.S. Application Data
(60) Provisional application No. 60/286,974, filed on Apr. 30, 2001.
(60) Provisional application No. 60/330,586, filed on Oct. 25, 2001.

(51) Int. Cl. A61M 5/32

(52) U.S. Cl. 604/198; 128/917

(57) ABSTRACT

A catheter device includes a catheter tube having a bend and having a distal end for insertion into the body of an animal or human and a proximal end. The catheter device also includes a sealing member located on the outside of the catheter tube in the vicinity of the bend. The sealing member prevents the leakage of fluid from the catheter tube when the catheter tube is pierced by the a needle in the vicinity of the bend. Preferably the catheter device is made by an injection molding process. Also disclosed is a combination needle and safety guard that includes: a needle having a sharpened distal end and proximal end; a safety guard having a proximal end and a distal end. The distal end of the needle extends beyond the distal end of the safety guard in a direction away from the proximal end of the safety guard when the needle is in use or prior to use. The safety guard further includes: a needle shield located in the vicinity of the distal end of the safety guard and fixed relative to the safety guard; and a biasing member adapted to move the distal end of the needle relative to the needle shield. When the distal end of the needle is retracted in a direction toward the proximal end of the safety guard, the needle shield prevents movement of the needle beyond the needle shield due to the relative movement of the needle relative to the needle shield.

### BACKGROUND OF THE INVENTION

1. Field Of The Invention

The present invention relates to catheters and catheter safety guards, preferably made from an injection molded plastic material.

2. Description of the Known Art

Catheters are known in the art. Prior to the present invention, there have been problems relating to catheters including the difficulty of removing the needle after the catheter has been inserted into the body of an animal or human without disconnecting the catheter tube. In addition, there have been problems of needle stick injuries caused by used, contaminated needles. Known catheters and guards are described in U.S. Pat. Nos. 5,810,780 (Brimhall et al.), 5,743,891 (Tolkoff et al.), 5,697,914 (Brimhall et al.), 6,299,602 (Miller et al.), 5,690,619 (Erskine), 5,300,045 (Plassche, Jr.), and 6,162,195 (Igo et al.).

### SUMMARY OF THE INVENTION

One object of the invention is to overcome the disadvantages of the known art described above. Another object of the invention is to provide a catheter that allows the needle to be removed with less difficulty after insertion into a body. Yet another object of the invention is to provide a catheter safety guard that reduces the danger of accidental needle stick injuries after removal from a body. Still another object of the invention is to provide an improved method of manufacturing a catheter and safety guard. Another object of the invention is to provide an efficient and inexpensive method for inserting a catheter into the body, usually into a vein or an artery, and being able to get dispose of the needle without disconnecting the catheter tube from what it is connected to, e.g., a blood bag or a container of medicine.

Yet another object of the invention is to provide an attached strap for attaching the catheter to the limb or part of the body where it is used. A further scope of the invention is to provide a needle that is elastically loaded so that when it is removed from the catheter it seals behind the needle, leaving the catheter tube with free access. A further part of the scope of the invention is a needle with an attached mechanism that when the needle is removed from the catheter the mechanism acts as a safety point dropping over the needle.

In order to achieve the foregoing and further objects, there has been provided according to one aspect of the invention a catheter device that includes: a catheter tube having a bend and having a distal end for insertion into the body of an animal or human and a proximal end; and a sealing member located on the outside of the catheter tube in the vicinity of the bend. The sealing member prevents the leakage of fluid from the catheter tube when the catheter tube is pierced by the a needle in the vicinity of the bend.

According to another aspect of the invention there has been provided, a combination needle and safety guard that includes: a needle having a sharpened distal end and proximal end; a safety guard having a proximal end and a distal end, wherein the distal end of the needle extends beyond the distal end of the safety guard in a direction away from the proximal end of the safety guard when the needle is in use or prior to use and the safety guard further comprises; a needle shield located in the vicinity of the distal end of the safety guard and fixed relative to the safety guard; and a biasing member adapted to move the distal end of the needle relative to the needle shield. When the distal end of the needle is retracted in a direction toward the proximal end of the safety guard, the needle shield prevents movement of the needle beyond the needle shield due to the relative movement of the needle relative to the needle shield.

Further objects, features and advantages of the present invention, will become readily apparent from detailed consideration of the preferred embodiments which follow.

### BRIEF DESCRIPTION OF THE DRAWINGS

FIG. 1 depicts a catheter device having a needle inserted and safety guard according to a preferred embodiment of the invention.

FIGS. 2a-2b depict a safety guard according to a preferred embodiment of the invention.

FIG. 3 depicts a catheter device having a needle inserted according to another preferred embodiment of the invention.

FIG. 4 depicts a catheter device and a combination of needle and safety guard according to another preferred embodiment of the invention.

FIG. 5 depicts a safety guard with the needle in a retracted position according to another preferred embodiment of the invention.

FIG. 6 depicts a safety guard with the needle in a retracted position according to another preferred embodiment of the invention.

FIG. 7 depicts a safety guard according to another preferred embodiment of the invention.

FIG. 8 depicts a catheter device and a combination of needle and safety guard according to another preferred embodiment of the invention.

### DETAILED DESCRIPTION OF THE PREFERRED EMBODIMENTS

One aspect of the invention provides an improved catheter device, that is preferably made from an injection-molded plastic. The device includes a catheter tube, preferably a one-piece continuous catheter tube. The catheter tube includes a distal end for insertion into the body of an animal or human and a proximal end that can be connected to a drainage collection, or a container with medicine or blood for delivery to the body. The catheter tube has a bend sufficient to allow the needle to pass through the wall of the tube at the bend. The bend is preferably in the range 90 degrees to 170 degrees, most preferably about 160 degrees. The angle is measured from the distal end of the catheter tube as shown in FIG. 1 as θ. The bend can be imparted during the manufacture process, or alternatively at a point after manufacturing.

The catheter device also includes a sealing member. The sealing member is in proximity to the bend, and preferably is in contact with it, and more preferably formed intimately in contact with the catheter tube as is possible by injection molding described in more detail below. The function of the sealing member is to prevent leakage of fluid from the catheter tube after the needle is withdrawn. This is described more fully below. The sealing member is preferably an elastomer, such as septic rubber. However, the sealing member can also include any other material that is capable of sealing after withdrawal of a needle. Other examples could include layers of paper, or any material that forms a water sealable mass upon contact with a fluid, such as cellulose fibers or silicone gels.

The catheter device can also include a tie-strap for anchoring the catheter to the body, such as tying the catheter to the limb (arm or leg) of a human patient. The tie-strap can be made of any suitable material such as plastic, rubber, rope, etc. The tie-strap is attached to the catheter device by any suitable means, such as adhesive, etc. In a preferred embodiment described below, the tie-strap can be molded at the same time, or present in a mold at the time the catheter device is formed by injection molding.

As noted above, in a preferred embodiment, the catheter device is manufactured by a method that includes an injection molding operation. Specifically, a preferred method, although not the only method of making the catheter device, is to insert a proximal end of the needle with a sharpened point, or a proximal end with a lead point on it, into the end of the tube that will be inserted into the body, hereinafter called the distal end of the tube. The tube, at a desired length from the distal end of the tube, is bent as described above. When the proximal end of the needle reaches the bend in the tube, the needle punches or pierces the tube from the inside to the outside. An advantage of piercing the tube from the inside to the outside is that this direction of piercing will cause the puncture debris to end up on the outside or stretch to the outside when the hole is punched. The needle then passes through a sealing member, and then on to the point where the sharpened distal point of the needle is in the vicinity of and preferably extends a small amount out of the distal end of the catheter tube. The catheter tube with the sealing member and the needle in place is then put into an injection mold, plastic is then molded over the top, encasing the entire device.

The tie-strap can be molded at the same time the plastic is injected around the catheter device, or separately attached to the mold and encased with plastic during the injection molding operation. Of course, the ends of the tie-strap should remain free in order to allow attachment to the body of an animal.

The device with the needle in place is then removed from the mold. The proximal end of the needle preferably includes a member that provides cover or protection at that proximal end or facilitates manipulation of the needle and/or to prevent injury if the proximal end of the needle is sharpened.

The catheter device is then ready for use, usually after sterilization. In use, the distal end of the needle extending a small distance from the distal end of the catheter is used to facilitate insertion of the catheter into the desired portion of the body, such as by piercing a blood vessel. When the distal end of the catheter is inserted into the artery, the needle is then retracted, which allows the blood to flow or the medicine to begin flowing into the body. The needle is retracted such that the distal end of the needle will pass through the catheter tube at the bend. When the distal end of the needle is pulled from the sealing member, the hole formed by the presence of the needle is sealed between the catheter and the needle hole, such that little, and preferably no, fluid is leaked from the catheter.

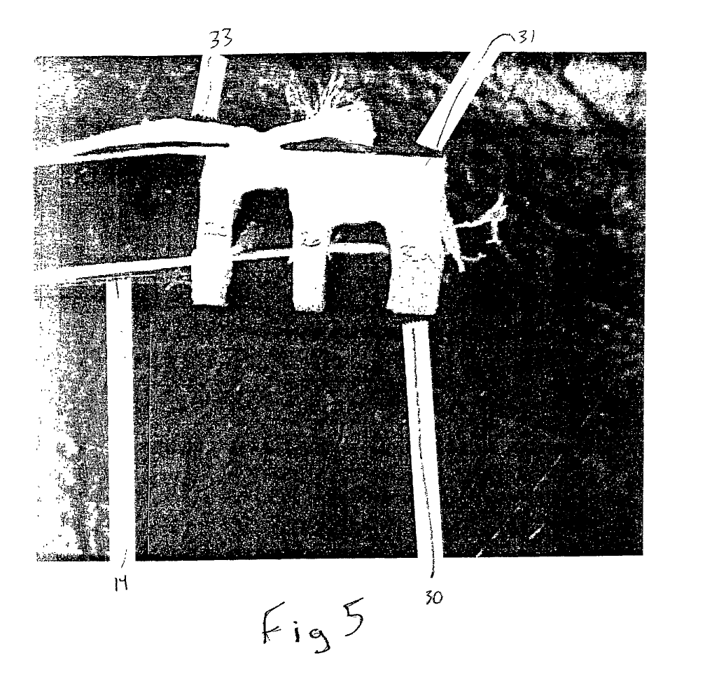

The catheter device is now described with reference to the embodiment shown in FIG. 1. The catheter device 10 in FIG. 1 includes catheter tube 11 that includes a proximal end 11a and distal end 11b. The bend in the catheter tube is shown as 12 and the angle of the bend (θ) is shown. The sealing member 13 is, in this embodiment, a sealing elastomer. The catheter device also includes needle 14 having a proximal end 14a and distal end 14b. Proximal end of needle 14a also includes a member 15 to allow manipulation of the needle. FIG. 3 also shows a catheter device according to a preferred embodiment of the invention. Tie-strap 16 is also shown.

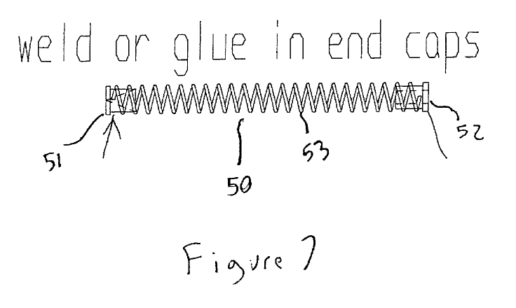

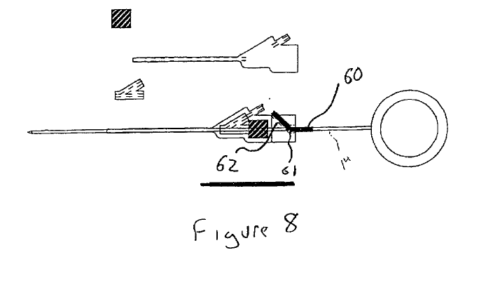

According to a related aspect of the invention, a safety guard is also provided that reduces the risk of needle stick injuries after use. The safety guard can be used together or separately from the catheter device described above. In a preferred embodiment, the safety guard is used together with the catheter device. In a preferred embodiment, the safety guard is formed by an injection molding process. In the broadest aspect, the safety guard has a proximal and distal end. Before and during use, the distal end of the needle extend beyond the distal end of the safety guard. Located in the vicinity of the distal end of the safety guard is a needle shield that is fixed relative to the safety guard. The function of the needle shield is to extend into the path of needle (either by movement of the shield or the needle) after the distal end of the needle has been retracted from the catheter device or other device to prevent the distal end of the needle from sticking a person or anything else not intended to be stuck with the needle. The needle shield can be made from any material that is capable of preventing the distal end of the needle from moving forward beyond the needle shield, such as by piercing the needle shield. For example, the needle shield can be plastic, metal, ceramic etc. In a preferred embodiment, the needle shield and safety guard are a one piece unitary structure such as shown in FIGS. 1 and 4 to 6. Preferably, the one piece unitary structure is injection molded. In other embodiments, the needle shield may be made from a different material as shown in FIG. 7.

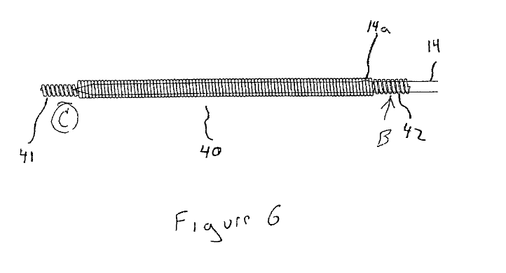

To move the needle relative to the needle shield, a biasing member is used. The biasing member may be used to either move the needle into alignment with the needle shield, or move the needle shield into alignment with the needle. Of course, since the needle shield is fixed relative to the safety guard, the safety guard will also move relative to the needle. The biasing member can include a spring, elastomer or the like to act on the needle to push the needle relative to the needle shield, or to act on the needle shield relative to the needle. The biasing member can also be part of the safety guard structure as explained in connection with FIGS. 4 and 5 below.

The safety guard can also include a needle guard that is located in behind the needle shield in a direction toward the proximal end of the safety guard. The needle guard can include any structure that is capable of grasping or binding the needle after the needle has been retracted from the catheter device or other device. In a preferred embodiment, the needle guard is a structure that has a surface area or cross-sectional area perpendicular to the longitudinal axis of the needle. The surface has an elongated slot extending through the surface, having a diameter at one end of the slot that is larger than the diameter of the needle and a diameter at the opposite end of the slot that is smaller than the diameter of the needle. The needle extends through the elongated slot. In use, the when the needle is retracted a biasing member (that may be the same or different than the biasing member described in connection with the needle guard) pushes the needle toward the smaller diameter of the slot. The side of the slot comes into contact with the needle shaft and prevents further movement of the needle due to the friction force between the sides of the slot and the needle shaft. In a preferred embodiment, the structure is a planar structure such as a disc that has its major surface area perpendicular to the longitudinal axis of the needle.

The safety guard can also include a tether device, such as a tether line to prevent the distal end of the needle from being retracted out of the safety guard in a direction away from the needle shield. This feature can be used together or separately from needle guard described above to prevent further retraction of the needle. The tether device has one end attached to the safety guard and another end attached to a portion of the needle, for example, the member that covers or protects the proximal end of the needle. The tether device allows the needle to be freely extended for use (assuming the needle shield is not in the path of the needle) and allows the needle to be retracted a predetermined length before the tether device arrests retraction.

In a preferred embodiment, the safety guard can include an interlocking device to connect it to the catheter device or any other device the needle guard is being used with, such that movement between the safety guard and catheter or other device is prevented. For example, the fastener can include a tab and slot arrangement, or interconnecting tabs.

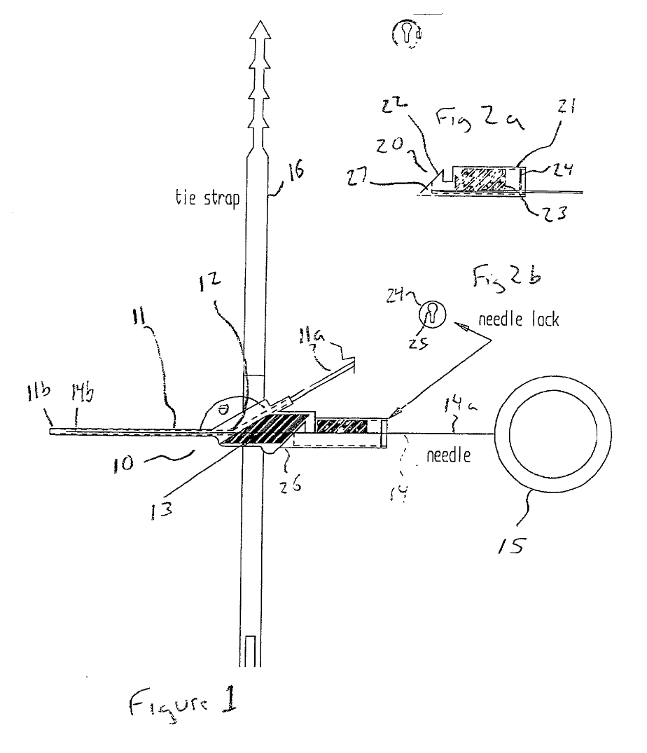

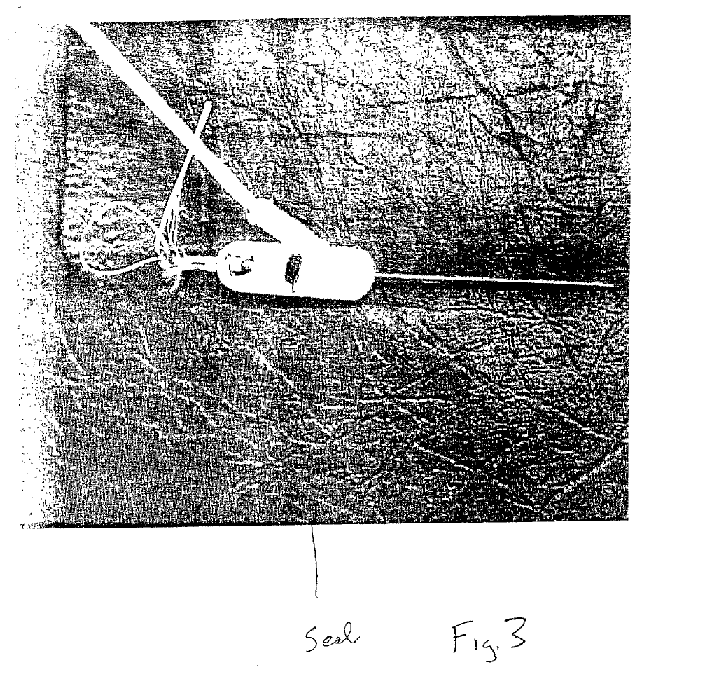

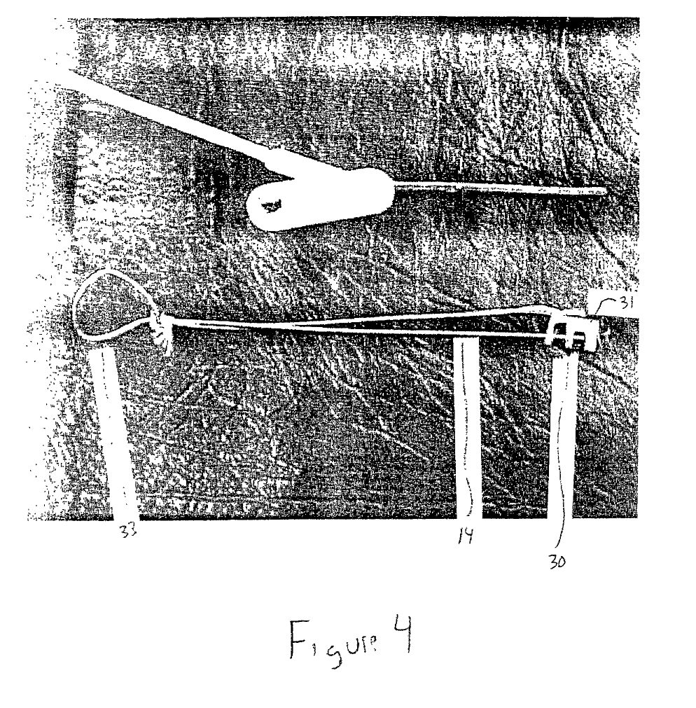

The safety guard is now described with reference to the embodiments shown in FIGS. 2, 4 and 5. In the embodiment shown in FIG. 2a, safety guard 20 includes a tube 21 formed from plastic. In this embodiment, the needle shield is a piece of material 27 that has a surface or cross-sectional area substantially perpendicular to the longitudinal axis of the needle with a hole in the distal end 22 for the needle to pass through the center of the tube 21. Biasing member (an elastomer in this instance) 23 is placed above the needle 14 in a state of compression. As shown in FIG. 2a, the safety guard in this embodiment includes disc-shaped needle guard 24. Needle guard 24 includes a key-hole shaped slot 25 that includes a larger diameter at one end of the slot and a smaller diameter at the other end of the slot. Needle guard 24 can be made of any material, such as plastic (e.g., polypropylene) or metal. The needle guard is then placed in the proximal end of the safety guard as shown in FIG. 2a.

To hold the safety guard in place, the safety guard can include over mold 26 as shown in FIGS. 1 and 2a. In use, the needle is then retracted so that the safety guard may be inserted into the body of the catheter device. The needle is then placed into the safety guard with elastomer on the top. The key-hole shaped needle guard is then placed on the needle. When the catheter is inserted into the artery, the needle is then retracted, which allows the blood to flow or medicine or other fluid to flow. When the needle is pulled from the elastomer seal, the hole in the seal is plugged. When the needle clears the plastic overmold, the safety guard drops from the catheter device holder, and the elastomer that is under compression on top of the needle forces the needle down into the safety guard and also into the needle shield. When the needle is forced downward, it tends to bind on the needle guard, stopping the needle from going back further. When the needle is forced to the bottom of the tube as shown in the embodiment according to FIG. 2a, the point of the needle is behind a thick piece of plastic needle shield 27, so the needle is now in a safety mode. A person handling the needle and safety guard cannot push it forward or easily push it backwards. It can then be thrown away, and it will not accidentally puncture someone else.

In the embodiment shown in FIGS. 3 to 5, the safety guard 30 includes an elongated member 31 that is substantially parallel to the longitudinal axis of the needle 14. In this embodiment, the elongated member is made of a flexible elastic material that has a memory to retain its original shape after being flexed. The safety guard 30 also includes three members 32a-c extending away from the elongated member 31. Member 32a is the needle shield that has a hole to allow the needle to pass through. Members 32b and 32c located away from member 32a in the proximal direction of the safety guard have holes therein to allow the needle 14 to pass through. In use, the elongated member 31 is slightly flexed resulting in the needle shield 32a being biased against the needle 14. When the needle 14 is retracted from the hole in needle shield 32a, the elongated member straightens causing the needle shield 32a to shift in a direction perpendicular to the axis of the needle 14. This results in the hole of needle shield 32a no longer being aligned with the needle, thus preventing forward movement of the needle. The safety tether shown in FIGS. 4 and 5 prevents the needle from being retracted out of the safety guard.

FIG. 6 shows another embodiment of the safety guard according the present invention. In this embodiment, the safety guard 40 is a continuous spring 43. The spring has reduced ends 41 and 42. Reduced end 41 is located at the distal end of the spring and functions as the needle shield, when the needle is in the retracted position. Reduced end or needle shield 41 is reduced to prevent needle 14 from extending beyond needle shield 41 in a distal direction. The other end of the spring has reduced end 42 that can function as a needle guard. In this embodiment, the dimensions of the reduced end 42 are sufficiently large to allow the needle to move in a longitudinal direction. The needle 14 has a bulge 14a, which is dimensioned larger than the reduced end 42. The bulge 14a prevents the needle from moving toward the proximal end of the safety guard when bulge 14a abuts the reduced end 42. The needle is inserted by bending the spring and inserting the proximal end having the bulge through the gap in the coils of the spring at the point the spring is bent. As described above, the movement of the spring in the proximal direction will be limited by bulge 14a and needle guard 42. The biasing member is provided when the spring is flexed to allow insertion of the needle into the safety guard as described below.

In another embodiment depicted in FIG. 7, the safety guard 50 is similar to that shown in FIG. 6. In this embodiment, the reduced ends 51 and 52 are provided by end caps that are welded or glued into the ends of the spring 53. The cap 51 is dimensioned such that the sharpened, distal end of the needle (not shown) will not be able to extend beyond cap 51. The cap 52 is dimensioned such that the bulge 14a of the needle described in connection with FIG. 6 will stop movement toward the proximal end when the bulge 14a abuts the end cap 52. The end caps may be of any suitable material, such as a plastic or metal.

The use of the safety guard described in FIGS. 6 and 7 is shown in FIG. 8 in connection with the catheter device. In this embodiment, the spring is shown as 60. The spring is flexed at point 61 to allow insertion of the needle 14 as described above. The catheter device includes a circular, elongated hole 62 that is at an angle, such as 20-80, preferably about 45 degrees relative to the longitudinal axis of the spring. This hole allows the bent distal portion of the spring to be inserted into it. When the needle is retracted in a proximal direction, the distal sharpened end of the needle must first be retracted beyond the point 61 the spring is flexed at. When the needle is retracted beyond point 61, the spring is likewise retracted from the hole and resumes its linear shape such that the needle 14 will be within the coils of the spring.

While a number of preferred embodiments of the present invention have been described, it should be understood that various changes, adaptations and modifications may be made therein without departing from the spirit of the invention and the scope of the appended claims. As used herein and in the following claims, articles such as “the,” “a” and “an” can connote the singular or plural.

1. A catheter device comprising:
a catheter tube having a bend and having a distal end for insertion into the body of an animal or human and a proximal end; and
a sealing member located on the outside of the catheter tube in the vicinity of the bend, wherein said sealing member prevents the leakage of fluid from the catheter tube when the catheter tube is pierced by a needle in the vicinity of the bend.

2. A catheter device according to claim 1, wherein the sealing member is an elastomer.

3. A catheter device according to claim 1, wherein the sealing member is in direct contact with the catheter tube in the vicinity of the bend.

4. A catheter device according to claim 1, wherein the bend is in the range of 90 degrees to 170 degrees.

5. A catheter device according to claim 2, wherein the sealing elastomer is directly in contact with the catheter tube.

6. A catheter device according to claim 2, further comprising a needle having a sharpened distal end and a proximal end, wherein the needle extends through the sealing elastomer, such that the distal end of the needle is located in the vicinity of the distal end of the catheter.

7. A catheter device according to claim 6, wherein the distal end of the needle extends beyond the distal end of the catheter tube.

8. A catheter device according to claim 6, further comprising a tie-strap for anchoring the catheter device to the body.

9. A catheter device according to claim 2, further comprising an injection molded plastic material injection molded around at least a portion of the catheter tube and sealing elastomer to encase at least a portion of the catheter tube and sealing elastomer.

10. A catheter device according to claim 8, further comprising an injection molded plastic material injection molded around at least a portion of the catheter tube, tie-strap and sealing elastomer to encase at least a portion of the catheter tube and sealing elastomer.

11. A method of making a catheter device, comprising:
providing a catheter tube having a bend and having a distal end for insertion into the body of an animal or human and a proximal end;
providing a sealing member located on the outside of the catheter tube in the vicinity of the bend, wherein said sealing elastomer prevents the leakage of fluid from the catheter tube when the catheter tube is pierced by a needle in the vicinity of the bend;
providing a needle having a sharpened distal end and a proximal end, wherein the needle extends through the sealing member, such that the distal end of the needle is located in the vicinity of the distal end of the catheter;
inserting the catheter tube, sealing member, and needle into a mold;
injection molding a plastic around at least a portion of the sealing member, catheter tube and needle to encase as least a portion of the of the sealing member, catheter tube and needle in the plastic.

12. A method according to claim 11, wherein the bend is in the range of 90 to 170 degrees.

13. A method according to claim 11, wherein the sealing member is an elastomer.

14. A method according to claim 11, wherein the step of providing the needle further comprises:
inserting the proximal end of the needle having a sharpened proximal end or a proximal end having a lead point into the distal end of the catheter tube up to the bend in the catheter tube;
piercing the proximal end of the needle through the catheter tube and through the sealing elastomer until the distal end of the needle is located in the vicinity of the distal end of the catheter.

15. A method according to claim 11, further comprising the step of providing a tie-strap for anchoring the catheter to the body, and wherein the step of injection molding includes encasing at least a portion of the tie-strap in the injection molded plastic.

16. A combination needle and safety guard comprising:
a needle having a sharpened distal end and proximal end;
a safety guard having a proximal end and a distal end, wherein the distal end of the needle extends beyond the distal end of the safety guard in a direction away from the proximal end of the safety guard when the needle is in use or prior to use, and the safety guard further comprises;
a needle shield located in the vicinity of the distal end of the safety guard and fixed relative to the safety guard; and
and a biasing member adapted to move the distal end of the needle relative to the needle shield, whereby when said distal end of the needle is retracted in a direction toward the proximal end of the safety guard, the needle shield prevents movement of the needle beyond the needle shield due to the relative movement of the needle relative to the needle shield.

17. A combination needle and safety guard according to claim 16, wherein the safety guard further comprises a needle guard located behind the needle shield in a direction toward the proximal end of the safety guard.

18. A combination needle and safety guard according to claim 17, wherein the needle guard comprises a structure having a surface or cross-section area perpendicular to the longitudinal axis of the needle with an elongated slot having a diameter at one end of the slot that is larger than the diameter of the needle and a diameter at the opposite end of the slot that is smaller than the diameter of the needle and said needle extends through the elongated slot.

19. A combination needle and safety guard according to claim 18, further comprising a biasing member for biasing the needle in the direction of the smaller diameter of the needle guard when the distal end of the needle is retracted in a direction toward the proximal end of the needle guard.

20. A combination needle and safety guard according to claim 19, wherein the safety guard is in the shape of a tube and said needle passes through the tube.

21. A combination needle and safety guard according to claim 20, wherein the needle shield has a cross-section that blocks on the distal end of the tube having an opening to allow the needle to pass through, whereby when the needle is retracted through the opening in a direction toward the proximal end of the tube, the biasing member pushes the distal end of the needle in a direction away from the opening.

22. A combination needle and safety guard according to claim 21, wherein the biasing member is an elastomer positioned in the tube, whereby the needle compresses the elastomer when the needle extends through the opening in the needle shield.

23. A combination needle and safety guard according to claim 21, wherein the safety guard further comprises a needle guard located behind the needle shield in a direction toward the proximal end of the safety guard and the needle guard comprises a material having a cross-section area perpendicular to the longitudinal axis of the needle with an elongated slot having a diameter at one end of the slot that is larger than the diameter of the needle and a diameter at the opposite end of the slot that is smaller than the diameter of the needle and said needle extends through the elongated slot.

24. A combination needle and safety guard according to claim 23, wherein the biasing member biases the needle in the direction of the smaller diameter of the needle guard when the distal end of the needle is retracted in a direction toward the proximal end of the needle guard.

25. A combination needle and safety guard according to claim 24, wherein the biasing member is an elastomer positioned in the tube, whereby the needle compresses the elastomer when the needle extends through the opening in the needle shield.

26. A combination needle and safety guard according to claim 16, wherein the safety guard further comprises an elongated member having at least a portion that is substantially parallel to the longitudinal axis of the needle, and at least one member extending away from the elongated member said one of the members being located in the vicinity of the proximal end of the safety guard, and said one member having an opening for the needle to pass through.

27. A combination needle and safety guard according to claim 26, wherein the needle shield comprises a further member extending away from the elongated member having an opening to allow the needle to pass through, whereby when the needle is retracted through the opening in a direction toward the proximal end of the safety guard, the biasing member move the distal end of the needle relative to the needle shield in a direction away from the opening.

28. A combination needle and safety guard according to claim 27, wherein the biasing member an elastic, flexible portion of the elongated member that is deflected when the needle passes through the opening of the needle shield.

29. A combination needle and safety guard according to claim 16, wherein the safety guard is a spring.

30. A combination needle and safety guard according to claim 29, wherein the needle shield comprises the distal end of the spring having a reduced diameter that is smaller than the diameter of the needle, and the biasing member is a portion of the spring that is deflected to allow the needle to pass through the coils of the spring.

31. A combination needle and safety guard according to claim 30, wherein the safety guard further comprises a needle guard located behind the needle shield in a direction toward the proximal end of the safety guard, wherein the needle guard comprises the proximal end of the spring having a reduced diameter, and the needle comprises a section having a diameter that is greater than the proximal end of the spring having the reduced diameter.

32. A combination needle and safety guard according to claim 31, wherein the distal and proximal ends of the spring having a reduced diameter comprise end caps located at the ends of the spring.

33. A combination needle and safety guard according to claim 31, wherein the distal and proximal ends of the spring having a reduced diameter comprise a portion of the spring having reduced diameter coils.

34. A combination needle and safety guard according to claim 16, wherein the needle shield and safety guard are a one-piece injection molded plastic.

35. A combination catheter device, needle and safety guard comprising:
a needle having a sharpened distal end and proximal end;
a catheter device comprising:
a catheter tube having a bend and having a distal end for insertion into the body of an animal or human and a proximal end; and
a sealing member located on the outside of the catheter tube in the vicinity of the bend, wherein said sealing member prevents the leakage of fluid from the catheter tube when the catheter tube is pierced by the needle in the vicinity of the bend; and
safety guard having a proximal end and a distal end, wherein the distal end of the needle extends beyond the distal end of the safety guard in a direction away from the proximal end of the safety guard when the needle is in use or prior to use and the safety guard further comprises;
a needle shield located in the vicinity of the distal end of the safety guard; and
and a biasing member adapted to move the distal end of the needle relative to the needle shield, whereby when said distal end of the needle is retracted in a direction toward the proximal end of the safety guard, the needle shield prevents movement of the needle beyond the needle shield due to the relative movement of the needle relative to the needle shield.
